(19) United States
(12) Patent Application Publication
(10) Pub. No.: US 2010/0196518 A1
(43) Pub. Date: Aug. 5, 2010

(54) Method and Device for the Treatment of Water for Purposes Conducive to Good Health

(76) Inventor: Peter Derkx, Rohrbronn (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/698,393

(22) Filed: Feb. 2, 2010

(30) Foreign Application Priority Data
Feb. 2, 2009 (DE) 10 2009 007 153.9

(51) Int. Cl. A61K 36/00; F17D 1/16; E03B 5/00; A61P 1/16; A61P 13/12; A61P 17/06

(52) U.S. Cl. 424/725; 137/13; 137/565.17

(57) ABSTRACT

This invention therefore presents a method and a device for the treatment of drinking water for purposes conducive to good health and the production of wellness and healthcare products. In this method the water to be treated is heavily swirled within a container (10). Using a rotor (50) with at least one impeller (54; 56) the water is put into rotating motions between the container's wall (18) and the container's center while being diverted through stream-leading installed parts (60; 70; 80). These installed parts also contain a cap (60) covering the impeller. In the direction of rotation forced by the impeller (54; 56) the water floats outwards and alongside the inner wall of the container (18). During the diversion, on its way back through the containers center, the water is forced into the reverse direction of rotation causing a heavy swirl close to the impeller.

The invention relates to a method and a device for the treatment of drinking water for purposes conducive to good health and the production of wellness and healthcare products. In detail, this invention relates to a method for the treatment of water, in which the water to be treated is heavily swirled within a container, a respective device for the treatment of water and the usage of the treated water for purposes conducive to good health.

Good drinking water is essential for life on earth. Predominantly, water can be seen as staple food but for all times it has also been used as a remedy. Lots of sanitaria rest their reputation on mineral springs—their spa waters are traditionally used for all kinds of medical conditions.

Recently, also drinking and mineral waters are spiked with supplements conducive to good health. In the market we can find these so called “wellness” products spiked with vitamins, minerals or plant extracts like e.g. tee-extracts.

Furthermore, there are also gas-enriched waters, mostly enriched with oxygen, which is added with pressure.

The so called “levitated”-waters are also treated waters. In this case, drinking water is mechanically heavily burdened with the objective of enriching the water with special physical qualities. The efficiency of such waters is discussed controversially as also the way of treating these waters is not defined. But it is indisputable that the swirling, which can occur while levitating water, increases the gas ratio within water. Air or oxygen can be added more easily through intensive stirring or swirling than simple sparging.

Basically, there is a constant need for good drinking water. Also, the strong pollution of waters leads increasingly towards the need for further treatment methods of supply water into good drinking water.

The invention is based on the task of finding a method for the mechanical treatment of drinking water which aerates the water mechanically and makes is receptible for gases and supplements. Due to its special quality this water shall be applicable for purposes conducive to good health.

This task is fulfilled by inventing a method for the treatment of water according to the attributes of claim 1, a device for the realization of the method according to the attributes of claim 8 and the usage according to claim 16.

Further beneficial developments of the invention are characterized in sub claims.

Water treated with the invention presents with an increased ability to solubilize gases, i.e. the solubility of a certain gas at a certain temperature is higher. This could be attributed to an enhanced “inner surface” due to the shear through high application of energy causing a division of water clusters. Other physical characteristics are also changed through the treatment. Among other things, compared with untreated water the treated water shows different crystallization images (see trial results below) and the swelling property of divers, especially organic substances and cells in treated water is improved.

According to current findings, the observed health-Improving effect of water treated with the invention could be that body cells are able to take in this water more easily.

The basically known heavy swirling for the treatment of water is realized in a special way for the method of this invention. The method is preferably carried out batch-wise. Therefore, one portion of water to be treated has to be given into the container. A rotor with at least one impeller, i.e. a propeller with blades pointing slightly towards the axis, swirls the water like a common blender. The water begins to rotate around the axis. At the same time rotating motions happen between the inner surface of the container and the container's center. Preferably, the container is arranged rotation-symmetrically around the rotor's axis.

According to the invention, the treatment method is developed in such a way that the water's flow is directed in a special way. By means of stream-leading installed parts in the container, as closer explained below, the water that is floating outwards and alongside the inner surface of the container in the direction of rotation forced by the impeller is on its way back through the container's center forced into the reverse direction of rotation causing a heavy swirling close to the impeller.

Preferably, the flow is partly accelerated additionally, on the one hand through narrowing the flow section down with installed parts with increasing bending—this method is particularly intended for the fins or blades which force the water's flow before or during the diversion into a random current along the container's wall. On the other hand through narrowing the flow section step-by-step, i.e. leading the water through a narrowed section in order to accelerate it immediately before it is diverted into the reverse direction of rotation. Both methods increase the swirling effect and divide the water extremely fine, up to its microstructures.

The treatment lasts preferably at least 2 (two) minutes, more preferably at least 5 (five) minutes.

For the acceleration and the diversion process of the water, enough energy has to be put in the water. Only after a certain treatment intensity becomes the water's change of physical characteristics obvious and stays stable long enough. In order to reach this, the rotor should be operated with at least 5000 rpm, preferably 8000 rpm, more preferably 10000 rpm. The necessary engine power depends on the water amount that needs to be moved. In one of the preferred ways of operation (see below) the engine power is 300 W up to 5.5 kW.

In one of the preferred ways of operation the filling volume, i.e. the charge volume for the method, is 1 up to 15 liters, preferably 1.5 up to 4 liters. At the moment, the filling volume of 2 up to 2.5 liters is especially preferred. The engine power for this amount should be at least 400 W.

Moreover, it is possible to feed or add gas during the treatment or into the freshly treated water. Therefore, because of the up-building pull effect caused by the swirling current, it is sufficient to let air or preferably oxygen stream into the container through an opening, e.g. in the container's cover. There are several breather holes in the container's cover. It is also possible to force the gas directly from a bottle, but then pressure compensation is necessary.

If desired, active ingredients or micronutrients, especially minerals, trace elements and/or vitamins can be added to the water. Both, adding gas and adding flavors and nutrients turns water into a healthcare product or a so called wellness product.

In principle, every drinking water or drinkable water is suitable for being treated with this invention. Drinking water of water works (tab water), spring water, mineral water or spa water can be used. Especially tab water should be additionally filtered preliminary to the treatment.

The special device for the realization of the invented method comprises a container with rotation-symmetrically conducted inner surface, at least one supply at least one drain, one engine-driven axially and with the rotational symmetry axis concentrically placed rotor and stream-leading installed parts. For easier cleaning purposes the device has been provided with a removable cover where installed parts or portions of these parts can be fixed to.

The rotor comprises at least one impeller (blade pointing towards the axis) preferably installed close to the bottom of the container. This impeller is covered by a bell-shaped stream-leading cap that leads up into a pipe or a cone whereas the cap and the pipe/cone are arranged coaxially around the rotor and container axis.

And at the upper end of the cone, above the filling level that needs to be observed while operating (filling level when not operated), there are stream-leading blade configurated installed parts which reverse the water's floating direction forced by the rotor. The deflecting blades or fins with preferably increasing bending in direction of flow are arranged annular at the upper brim of the pipe or the cone.

The rotor comprises an engine-driven shaft with at least one impeller. The (lower) impeller, respectively the (lower) propeller is preferably attached in the container's lower quarter or fifth, meaning close to the bottom. The same applies for the bottom edge of the cap. Preferably, the cap's bottom edge should be at least on one level with the impeller, i.e. the cap covers the impeller so far that at least half of it is within the volume provided by the cap. In an especially preferred embodiment the cap's bottom edge is scarcely below the only or the lower impeller.

In an especially preferred embodiment the rotor comprises two spaced axial propeller blades of which at least the upper one has to be covered by the cap. Preferably both impellers should be covered by the cap. The cap forces the downwards and outwards floating water to float with the impeller's/impellers' advance and direction or rotation upwards along the container's wall where it is diverted to the center avoiding a too early mixing of the different streams in the container.

When using two or more impellers they should preferably be installed shifted against each other.

The impellers are preferably installed in an angle between 3 and 6 degrees. Moreover, it is beneficial to slope the leading edge of the impellers' blades towards the direction of rotation, creating a cutting edge at the impeller's abutting face so the water is easily divided like cutting it with a knife. Through this, the water's antagonism is decreased.

As already mentioned above, the sidewall of the container on the level of the pipe/cone is reduced so that the circular gap between the container's inner surface and the pipe/cone is partly narrowed. Here, the reduced part is working as a ring-shaped die that accelerates the floating water at a point beneficial to the treatment.

The deflecting blades, which are part of the installed parts at the upper end of the pipe/cone, are increasingly curved in direction of the water's flow (cyclodically formed).

These parts can be linked with each other and/or to parts of the container or they can be assembled in the form of a framework between the container and its cover.

As surprisingly discovered, the treatment's product can be used for health care purposes or as a remedy. Assumedly, these health-beneficial effects are attributed to the fact that the water which physical characteristics have been improved through the treatment can be taken in by cells especially good.

The usage is amongst others reported for liver, kidneys, stomach, bowel, skin, eyes, heart, mucosa, genitals, the vascular system and lymph, especially also for type II diabetes, fresh wounds, headaches, migraine, edema, obstipation. The application can take place within the scope of treating a disease and medical condition or prophylaxis.

The changed structure of the treatment's product can be seen for example from its changed behavior in crystallization and the changed water-absorption of membranes and fleeces. For this see the following trials:

At first, the invention is described by means of illustrating an application embodiment. This application embodiment should only illustrate the invention and explain it better and should not be interpreted restrictively.

The figures show:

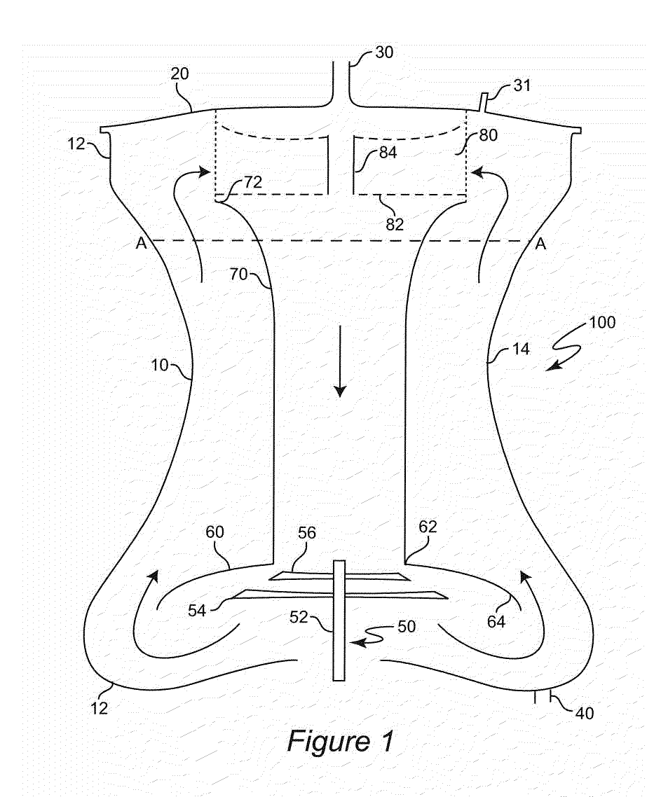

FIG. 1 the longitudinal section of the device;

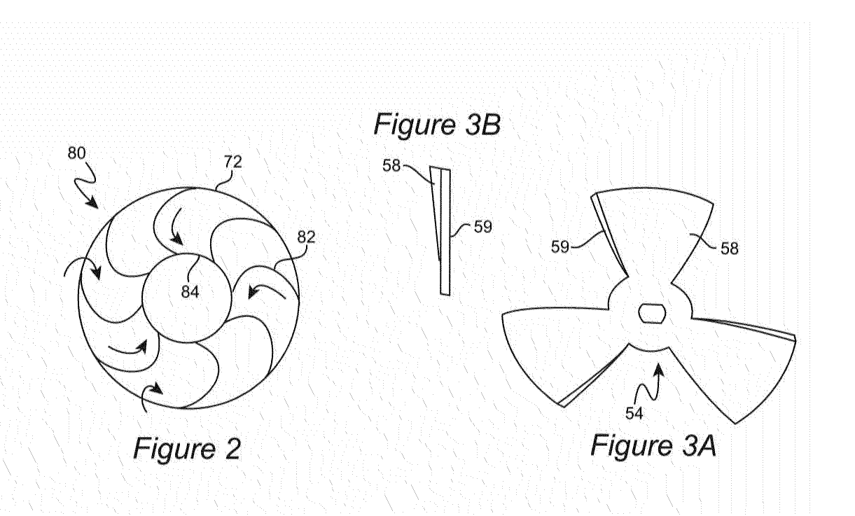

FIG. 2 paddle wheel, view from above (part of the device in FIG. 1);

FIG. 3 impeller (part of the device in FIG. 1),
a) view from above and
b) lateral view of one impeller;

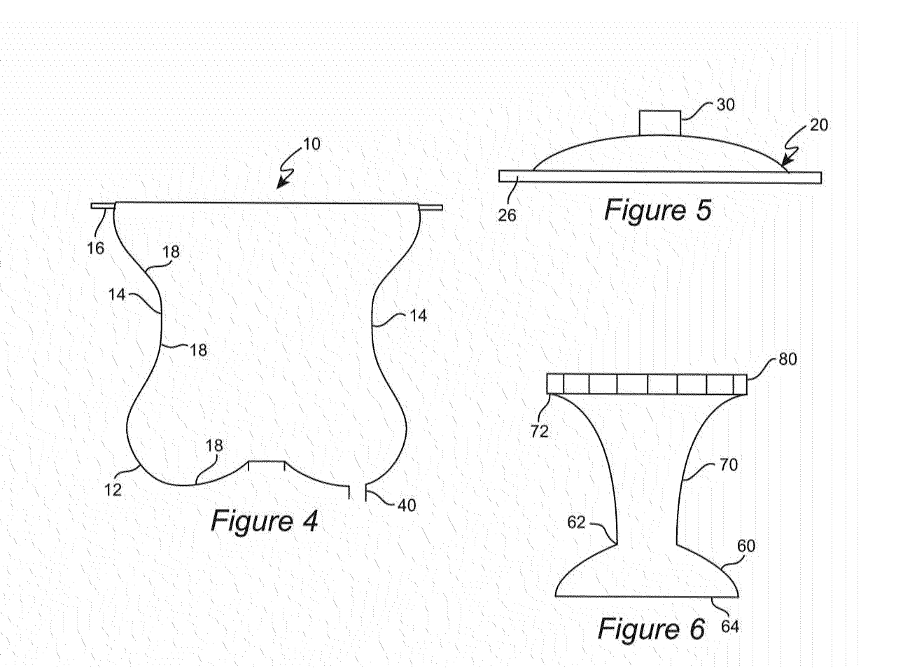

FIG. 4 the longitudinal section of the container (part of the device in FIG. 1);

FIG. 5 the cover, lateral view (part of the device in FIG. 1);

FIG. 6 the longitudinal section of the installed parts (part of the device in FIG. 1).

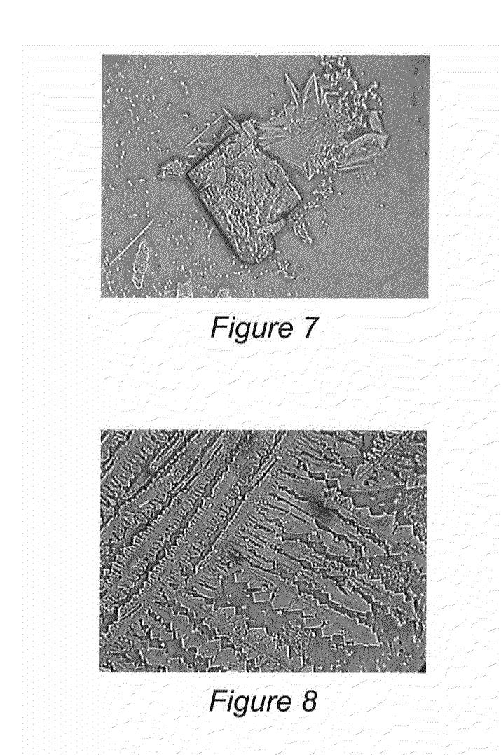

Subsequently, test results can be seen which characterize the treatment's product. The FIGS. 7 to 9 show drawings of:

FIG. 7 analysis pattern of a crystal—untreated tab water (water works Überlingen, Lake Constance);

FIG. 8 analysis pattern of a crystal—tab water treated with the invention (water works Überlingen, Lake Constance);

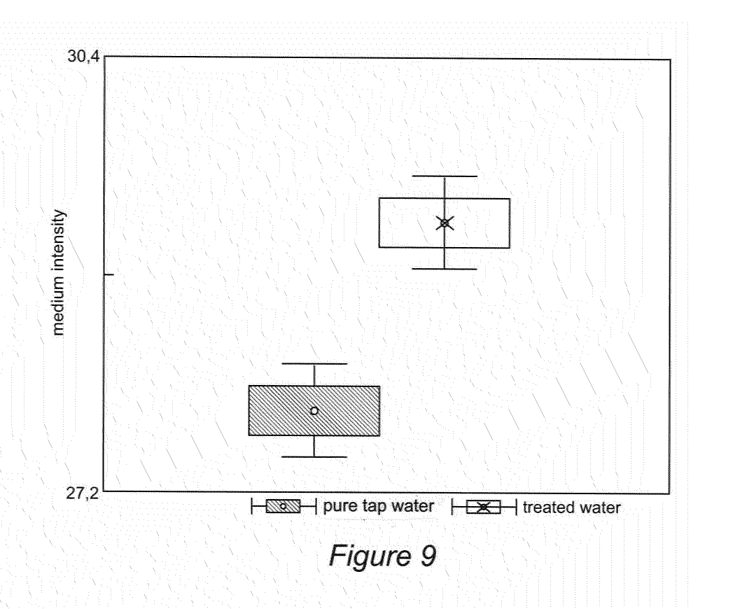

FIG. 9 chart regarding the test results of the Kirlian photography with treated and untreated samples.

FIG. 1 shows an application embodiment for a device 100 in the longitudinal section. A cup-shaped, rotation-symmetrically conducted and at the bottom hyperbolically formed container (10) is closed with a cover (20). On the cover there are two nozzles (30; 31) for the filling of the container, the gassing and degassing, the sampling and the insertion of sensors, thermometers and suchlike. If automatically filled through one pipe (not displayed), the nozzle 31 is used as feed.

As an alternative, the cover (20) can be removed and the container can be filled manually, especially in the case of smaller embodiments for 1 to 3 liters filling volume or in the case of non-automatic use. The treated water can be withdrawn through the drain (40). On the passage from bottom to the side wall the container (10) has a hyperbolical area (12) in order to divert the—by the rotor (50) swirled—water effectively.

Above this hyperbolical area and below the cups brim (12) the container's sidewall is reduced (14), narrowing the flow cross-section between the container (10) and the cone (70). The rotor consists of a rotor shaft (52) and two impellers (54 and 56). The rotor shaft (52) is driven by an engine not displayed in this figure. Both impellers (54; 56) of the rotor are covered by a cap (60) which is connected with a cone (70) at the upper end (62). The lower brim (64) of the cap (60) is lower than the impellers (54; 56). At the upper rim of the cone (72) follows a wheel (80) of deflecting blades, respectively fins, which is displayed in view from above in FIG. 2.

The container (10) is filled with water up to the maximum filling height (A). As soon as the engine is started and the rotor (50) starts rotating, the water streams underneath the cap (60) outwards and upwards. The floating directions of the water within the device are marked with arrows. While the water is floating up along the container's side wall it passes the narrowed area between the cone (70) and the reduced side wall (14) is accelerated and pushed upwards. Thrown towards the top the water encounters the paddle wheel (80) continues into the cone (70) which is fixed to the cover (20), and flows back down through the center of the container. The strongest swirling and the separation of the water masses happen at the upper brim of the cap (62), the section between cone (70) and cap (60).

In view from above FIG. 2 shows the paddle wheel (80) which consists of annular arranged deflecting blades (82). The deflecting blades (82) are kept together by a ring (84) and rest outside on the upper edge of the cone. Outside the paddle wheel (80) the water that is still rising within the direction of rotation forced by the rotor (50) floating over the edge of the cone (72) is reversed in its direction of rotation by the deflecting blades (80) and floats back through the cone's center—here vertical to the plane of drawing—towards the rotor. The direction of the water flow is displayed with arrows. The blades' radius of curvature (82) is continuously increasing with the water's direction of flow. Therefore, the blades are shaped, respectively curved, helically or in the form of a partial spiral.

FIG. 3 shows one of the impellers (54; 56) as a single part, in 3a) in view from above. The impeller has even three formed blades (58) with sloped (grounded) leading edges (59). These build knives which cut the water at the blade's frontal hem. FIG. 3b) shows the pitch and the leading edge (59) of a blade (58). The blades' angle in this example is 4°.

FIG. 4 shows a cup-shaped container (10), like in FIG. 1, as a single part without installed parts and rotor. This offers an especially good view on the hyperbolical area (12) and the reduced area (14) where the water is floating upwards along the container's inner side wall (18), respectively, in the circular outer streaming area. The cover is put on a flange (16) at the container's brim (12) which can be seen in lateral view in FIG. 5 (feed 31 is hidden). The respective flange of the cover has the number 26.

FIG. 6 shows the assembled installed parts, cap (60), cone (70) and paddle wheel (80). These parts are fix assembled (welded); however, they could also be detachably fixed to one another. The parts, which in this case build one connected part of the device 100, can have spacers and columns to be clamped between the container (10) and the respective cover (20). In this example however, the parts are fixed to the cover (20), i.e. either screwed or welded.

### Technical Details for the Example in a Special Embodiment

In an especially preferred embodiment of the invention the container (10) has a capacity of 2 to 2.5 liters up to the maximum filling level (A) and a total inner height of 165 millimeters between the upper edge and the bottom close to the shaft, 180 millimeters between the upper edge and the lowest point of the bottom. The container is (at the brim 12, inner side) 175 millimeters wide. The side wall of the container is approximately 25 millimeters reduced towards the center of the container.

The engine is an electric motor with 400 watts power with 2.7 ampere and a continuous output of 10,800 rpm. The distance between the bottom close to the shaft and the lower impeller (54) is 12 centimeters, the distance between the impellers (54; 56) is 10 millimeters.

The whole device is manufactured from high quality steel.

Test Results that Characterize the Treatment's Product:

1. Crystal-Analysis of the Treated Water

The treatment's product, i.e. water swirled according to the invention, was tested by means of a crystal-analysis.

Crystal-analysis is a popular method to test the biological quality, for example, of drinking water and was developed from the manufacturing technique of spagyrik medicines. Therefore, crystal salts from analysis material are displayed purely and analyzed under the microscope. For analyzing the quality of water, the water (without any supplements) is distilled at low temperatures. Then the distillation residue is ashed and calcinated (meaning thermal decomposition) and afterwards mixed with a defined volume of the distillate. One drop of the solution is put on a microscope slide and die liquid is evaporated at room temperature.

In this procedure the samples develop typical crystal images which give evidence for the quality of the water. These crystal images can be reproduced at any time from the same sample and always show the sample-specific crystal images.

FIG. 7 shows the crystallization image of a water sample of good, untreated tab water (water works Überlingen, Lake Constance); FIG. 8 shows—in the same magnification—the crystallization image of the same but with the invention treated water sample. The same tab water as in FIG. 7 was treated in the invention's device for 5 minutes and then analyzed like the sample from FIG. 7. Clear and repeatable differences in the structure can be seen.

Result: untreated water shows high amounts of cubical structures, derived from cuboids and cubes. Treated water shows tree and finning structures, hexagonal crystallization forms, snow crystal patterns.

2. Testing the Energy Content with the Kirlian Photography:

With another method, the Kirlian photography, the effect of the treated waters energy absorption was tested. The test showed that the treated water's radiation intensity of photons is significantly higher than the one of identical but untreated water.

The results are displayed in FIG. 9. The untreated tab water (box on the left) has a lower middle radiation intensity than the water treated with the invention (box on the right). For each group (treated, untreated) 4 measurements took place. The test results (number of occurrence, radiated photons) are statistically significantly different.

### Test Results for Health-Beneficial Effects and Field Reports:

Survey about the Impact of a drinking-water course with treated water on signs in patients suffering from Non-Alcoholic Steatohepatitis (NASH) according to papers of S. Francque, P. Michieösen, A. Ramon, A. Jung (at the Department for Gastroenterohepatology at the University Hospital Antwerp and the Diagnostic Genetic Laboratory in Cologne)

The non-alcoholic steatohepatitis is more and more seen as the cause of liver diseases and death. At present, there is no acknowledged definition for its causes and also no acknowledged therapy. It is assumed that the changes are caused by intestinal bacteria producing endotoxins which come through the cardio-vascular-system into the portal vein and then into the liver.

Here from the thesis arises that water treated with this invention can achieve a positive impact on the intestinal flora and therefore on the endotoxin-production. This would be beneficial for the treatment of NASH. Another assumption is that the liver is better able to detoxicate itself as the treated water can be better absorbed than regular water.

Patients and methods: The patients participating in this survey were all patients suffering from severe NASH.

Every day each patient was given at least 2 liters of treated water, not only as drinking water but also for the preparation of meals.

The patients were under constant observation—here the list of the things observed:
a. Compliance
b. Adverse effects
c. Anthropometry (body dimensions and weight, BMI)
d. Laboratory tests:
   1. Serum-Ferritin (intracellular iron-storage protein)
   2. Serum ALT (GPT) (alanine aminotransferase)
   3 Serum GGT (Gamma-Glutamyl-Transpeptidase)

These reference values are control parameters for the liver that are especially connected to the diagnostics of alcohol-induced (and non-alcoholic analog) liver damage.

The meaning of the chosen reference values:

Ferritin levels are considered as the mirror of iron deposits in the reticuloendothelial System (RES). Ferritin is a typical acute-phase protein—its level rise in case of Inflammation or tumors. It is the only parameter that makes it possible to clearly distinguish an iron deficiency anemia from anemias caused by tumors or infection.

GPT (ALT, alanine aminotransferase) is mainly based in mitochondria, but also in cytoplasm. An increased activity level in the serum is largely specific to liver diseases (parameter for the necrosis of liver cells).

Serum GGT (g-GT, Gamma-Glutamyl-Transpeptidase). Unlike other liver parameters γ-GT is bound to the cell membrane. γ-GT is specific to liver and bile ducts. Highest levels can be found in cases of cholestasis (icterus) and alcoholic hepatitis (diagnostics of alcoholism).

The level of the value is proportional to the extent of the liver damage.

### Results:

In total, five patients, three men and two women, have been analyzed. They were between 35 and 49 years old, their BMI (body-mass index) was between 35 and 37 kg/m².

a. Compliance:

Within the pilot study the compliance is evaluated as outstanding. This evaluation is based on patients' questionings and the daily record of the water used. It was enjoyable to drink the water.

b. Adverse Effects:

No adverse effects have been detected.

c. Anthropometry:

The patients' body weights (and therefore their BMI) stayed essentially constant for the period of the study (5 to 9 months).

d. Laboratory Tests:

Within the period of the study, a clear decrease of the measured parameters could be noticed:

Serum-Ferritin: 610±190 ng/ml→329±117 ng/ml with p=0.045

### Serum GPT: 50±6 U/l→38±6 U/l

### Serum GGT 46±22 U/l→39±8 U/l.

However, due to the low number of participants, these parameters achieved understandably statistically no significance.

Nevertheless, the decrease of the inflammation parameters is remarkable.

### Conclusion:

The pilot study provided the following results:

The treatment with the invention treated water led to a clear decrease of the parameters GPT and ferritin which are important indicators for active inflammations that are seen as the cause for non-alcoholic steatohepatitis. Therefore the conclusion is:
The treatment with this treated water leads to significant reduction of hepatitis and therefore to an improvement of the liver's status,
The treatment leads to a clear improvement of the inflammation indicators GPT and GGT,
These improvements cannot be traced back to the improvement of body weight or other parts of the metabolic syndrome, as the body weight stayed essentially constant,
Other probands who, for comparison, drank untreated tab water instead of treated water, experienced no positive effects,
There is a cause for guessing that other syndromes could be treated with the same method.

The study was conducted according to the Declaration of Helsinki (ethical requirements). The patients were selected according to NASH-criteria (clinical and laboratory criteria).

### Reports of Individual Patients:

1. Male patient >60 years old, hepatitis C patient after chemotherapy with chronic liver cirrhosis, on the list for liver transplantation, reports success:

He consumed daily 2 to 3 liters of the treated water (produced from local tab water, San Antonio, USA). After a few weeks his well-being improved significantly; the patient felt more energetic. The water was tolerated well, without flatulence or feeling of satiety. Also appetite and complexion improved. After 6 months the liver status had improved so much that the patient was no longer in need of a donor organ. Supported through athleticism like walking and calisthenics, which would have not been possible at the beginning of the treatment, the patient was able to rehabilitate his health completely. In the meantime, the water consumption was increased to approximately 3 to 4 liters a day. After 6 months the patient was able to do without any medication.
2. Female patient >34 years old, was diagnosed with “Lichen sclerosus et atrophicus”, complained about aches and pains and itching vaginal. She was treated by her dermatologist with cortisone salves, in the end with Clobetasol-17-propionate. The unbearable pain and itching recurred again and again; the salves only relieved the symptoms for a short amount of time and alternative methods, like bioresonance therapy, made no impact.
The patient began to drink 8 glasses of treated water per day (approximately 1.6 liters) produced from local tab water, Geldrop, in the Netherlands. Additionally, the patient performed daily vaginal irrigations with the treated water. Within 5 days the itching and pains improved, within ca. 6 weeks there was a significant improvement noticeable and after 3 months the symptoms were reduced to 10% of the initial symptomatology.
It is expected that corresponding treatments, like the one of patient 2, could also be efficient against psoriasis.

1. Method for the treatment of water whereby the water to be treated is provided in a container (10) and is swirled heavily, the methods comprises the step of putting water into rotating motions between the container's inner wall (18) and the container's center using a rotor (50) with at least one impeller (54;56); floating the water through stream-leading installed parts (60; 70; 80); and water floating outwards and alongside the inner wall of the container (18) in the direction of rotation forced by the impeller (54; 56) causing a heavy swirl close to the impeller as the water on its way back through the container's center is forced into the reverse direction of rotation.

2. The method according to claim 1, wherein the treatment lasts at least two minutes, preferably at least five minutes.

3. The method according to claim 1, wherein the rotor (50) is operated with at least 5000 rpm, preferably 8000 rpm, more preferably 10000 rpm.

4. The method according to claim 1, wherein the filling volume should be 1 up to 15 liters, preferably 1.5 up to 4 liters.

5. The method according to claim 1, wherein an additional gas is fed or added to the water, preferably oxygen.

6. The method according to claim 1, wherein active ingredients or micronutrients, especially Minerals, trace elements and/or vitamins are added to the water.

7. The method according to claim 1, wherein during the treatment before the diversion into the reserve direction of rotating the water has to flow through a narrow compartment within the container in such a way that it gains speed.

8. The device for the realization of the method according to claim 1 comprises a container (10) with rotation-symmetrically conducted inner surface, at least one supply (30; 31), at least one drain (40), one engine-driven axially placed rotor (50) and stream-leading installed parts (60; 70; 80) wherein the rotor (50) comprises at least one impeller (54; 56) (blades (58) pointing towards the axis).
This impeller is covered by a bell-shaped stream-leading cap (60) that leads up into a pipe or a cone (70) whereas the cap (60) and the pipe/cone (70) are arranged coaxially around the axis of rotor and container. And at the upper end of the cone (70), above the filling level (A) which needs to be observed while operating, there are stream-leading blade configurated parts (82) which reverse the water's direction of rotation forced by the rotor (50).

9. The device according to claim 8, wherein the rotor or at least one impeller of the rotor is installed close to the bottom.

10. The device according to claim 8, wherein the rotor (50) comprises two axially spaced impellers (54; 56), of which at least the upper one has to be covered by the cap (60).

11. The device according to claim 10, wherein the impellers (54; 56) are shifted against each other.

12. The device according to claim 8, wherein the impellers are installed in an angle between 3 and 6 degrees.

13. The device according to claim 8, wherein the leading edge of the impellers (54; 56) are sloped towards the direction of rotation.

14. The device according to claim 8, wherein the side wall of the container on the level of the pipe/cone (70) is reduced so that the circular gap between the container's inner surface (18) and the pipe/cone is partly narrowed.

15. The device according to claim 8, wherein the deflecting blades (82) at the upper end of the pipe/cone (70) are increasingly curved in direction of the water's flow.

16. The usage of this method according to claim 1 as remedy conducive to good health as well as the production of wellness and healthcare products.

17. The usage according to claim 16 as a healthcare product for the therapy of viscera and skin diseases, especially liver, kidney and skin diseases.
